SEQUENCE LISTING (4 sequences)

SEQ ID NO: 1 - protein, 10 aa, artificial
  peptide fragment containing MART-1 26-35 (A27L) epitope
ELAGIGILTV

SEQ ID NO: 2 - protein, 142 aa, artificial
  Constructed peptide containing ubiquitin sequence and four MART-1 mutant epitopes
MQIFVKTLTGKTITLEVEPSDTIENVKAKIQDKEGIPPDQQRLIFAGKQLEGRTLSDYNIQKESTLHLVLRLRGVVNSEFKHEELAGIGILTVAEFKSEELAGIGILTVAEEELAGIGILTVAEEELAGIGILTVAEEVNRA

SEQ ID NO: 3 - protein, 9 aa, artificial
  Peptide fragment containing the WT1 126-134 fragment
RMFPNAPYL

SEQ ID NO: 4 - protein, 9 aa, artificial
  Peptide fragment containing HIV-1 env gp120 90-98 fragment
KLTPLCVTL

(19) United States
(12) Patent Application Publication
(10) Pub. No.: US 2018/0078627 A1
(43) Pub. Date: Mar. 22, 2018

(54) METHOD FOR ANTIGEN LOADING OF DENDRITIC CELLS AND VACCINE

(71) Applicants: AGENCY FOR SCIENCE, TECHNOLOGY AND RESEARCH, Singapore (SG); TESSA THERAPEUTICS PTE. LTD., Singapore (SG)

(72) Inventors: Jieming ZENG, Singapore (SG); Shu WANG, Singapore (SG); Andrew KHOO, Singapore (SG)

(21) Appl. No.: 15/563,542

(22) PCT Filed: Mar. 28, 2016
(86) PCT No.: PCT/SG2016/050148

§ 371 Date: Sep. 29, 2017

(30) Foreign Application Priority Data
Mar. 31, 2015 (SG) 10201502560Q

(51) Int. Cl. A61K 39/00; C12N 5/0784; C12N 7/00; C07K 14/47; C07K 14/435

(52) U.S. Cl. CPC A61K 39/0011; C12N 5/0639; C12N 7/00; C07K 14/4748; C07K 14/435; A61K 2039/5154; C12N 2501/2304; C12N 2506/02; C12N 2740/15043; C07K 2319/95; A61K 2039/57; C12N 2501/22; A61K 2039/5156

(57) ABSTRACT

There is provided a method of loading antigen in a dendritic cell for antigen presentation, the method comprising: modifying a pluripotent stem cell with a nucleic acid molecule encoding an antigen or one or more immunogenic epitopes thereof; inducing the pluripotent stem cell to differentiate into a dendritic cell that expresses and presents the antigen or the one or more immunogenic epitopes thereof. Dendritic cells, vaccines and methods of using the dendritic cells and vaccines are also provided.

### CROSS-REFERENCE TO RELATED APPLICATION

This application claims benefit of, and priority from, Singapore provisional application No. 10201502560Q, filed on Mar. 31, 2015, the contents of which are hereby incorporated herein by reference.

### FIELD OF THE INVENTION

The present invention relates to methods of producing antigen-loaded dendritic cells and use of such cells in a vaccine.

### BACKGROUND

Dendritic cell (DC)-based vaccines are becoming a new therapeutic tool for treating cancer [1, 2]. This therapeutic strategy exploits the power and specificity of the immune system to fight cancer while at the same time avoiding the devastating and life-threatening side effects that often accompany traditional cancer therapies.

DC-based immunotherapy has a better safety profile and may provide better quality of life for cancer patients during treatment. However, it remains challenging to prepare high-quality DC vaccines in large enough quantities to induce clinically significant anti-cancer immunity due to the complexities in making such living cell products [3, 4].

Currently, most DC-based cancer vaccines are generated from a patient's own cells [6]. A large amount of peripheral blood mononuclear cells (PBMCs) are harvested from the patient via an invasive leukapheresis process. Monocytes are then isolated from PBMCs and differentiated into DCs. These monocyte-derived DCs (moDCs) are loaded with tumor antigens, matured and injected back to the patient. This production process is complicated and is subject to many technical and logistic difficulties. As well, the end products tend to be costly, as exemplified by the production of Dendreon's Provenge, the first ever FDA-approved DC-based vaccine for prostate cancer [7].

Currently, several antigen loading approaches have been used in DC vaccine production. Protein- or tumor lysate-loading provides the possibility to present multiple antigenic epitopes without being restricted by a subject's MHC haplotype. However, this approach requires a large amount of expansive clinical-grade tumor antigen protein or tumor cell lysate; moreover, the loaded tumor antigens tend to be presented by MHC class II rather than MHC class I [21].

Peptide-pulsing is a simple approach to load DCs with tumor antigen for presentation to CD8+ T cells, in which the MHC-restricted tumor antigenic peptides bind directly to the MHC class I molecule without going through the antigen processing pathways. However, these exogenous antigen-dependent approaches have short antigen presentation duration due to the high turnover rate of MHC/peptide complexes [22].

Nucleic acid-based antigen loading approach may extend tumor antigen presentation duration in DCs. In this approach, tumor antigen-coding DNA or RNA are delivered into DCs and the expression of these tumor antigen-coding nucleic acids may provide an endogenous supply of cytosolic tumor antigens that incline to be presented via endogenous pathway [23]. The antigen presentation efficiency using such approach depends largely on high-level transgene expression in DCs. For DNA-based antigen loading, viral vectors tend to be used [24]. For RNA-based antigen loading, tumor antigen-coding RNA can be delivered via electroporation into the DC cytoplasm, where the RNA is translated to produce tumor antigens. Unlike the DNA-based approaches, the RNA-based approach does not require a transcription step and thus is more efficient. However, the antigen presentation duration is limited by the poor stability and short lifespan of RNA [25].

From the standpoint of DC vaccine production, all the above-mentioned conventional antigen loading approaches require extra efforts to produce clinical-grade antigen payloads in various forms, such as peptide, protein, tumor cell lysate, DNA or RNA.

Additional manipulations to deliver these antigen payloads to DCs are mandatory. Such manipulations including cell incubation, transfection, electroporation and viral transduction, which tend to reduce the yield and viability of cells in a DC vaccine. Moreover, such manipulations need be repeated for every new batch of DC product, thus leading to batch-to-batch variability.

Furthermore, antigen loading of DCs is not a stand-alone step. Antigen loading must be done in coordination with ex vivo DC generation and maturation steps, which further complicates the whole DC vaccine production process for each batch of vaccine.

Aside from the technical and logistic difficulties associated with production, the quality of such patient-derived DC vaccine products can be highly variable due to uncontrollable patient-to-patient variation. Using these variable DC products in clinical trials makes it difficult to optimize critical parameters that are important for further improving vaccine efficacy. Moreover, such patient-derived DC products are often limited in supply, which makes it impossible to clinically evaluate the benefit of higher dosage and prolonged vaccination schedule.

To avoid the above-described issues associated with the use of limited and variable patient cells for DC vaccine production, it is necessary to explore other methods for producing antigen-loaded DCs.

### SUMMARY

In one aspect, there is provided a method of loading antigen in a dendritic cell for antigen presentation, the method comprising: modifying a pluripotent stem cell with a nucleic acid molecule encoding an antigen or one or more immunogenic epitopes thereof; inducing the pluripotent stem cell to differentiate into a dendritic cell that expresses and presents the antigen or the one or more immunogenic epitopes thereof.

The pluripotent stem cell may be an induced pluripotent stein cell, and may be stably modified with the nucleic acid molecule.

In the method, modifying may comprise transducing using a viral or nonviral method to deliver the nucleic acid molecule into the pluripotent stem cell. In some embodiments, the method of transducing may provide long-term transgene expression.

For example, modifying may comprise transducing the pluripotent stem cell with a retroviral vector, including for example a lentiviral vector.

The pluripotent stem cell may be a mammalian cell, including for example a human cell.

The antigen may be a full-length antigen, and may be a tumor antigen, a viral antigen, a bacterial antigen or an autoimmune disease antigen. The one or more immunogenic epitopes may be an epitope from a tumor antigen, a viral antigen, a bacterial antigen or an autoimmune disease antigen.

The nucleic acid molecule may further encode a targeting sequence fused to the antigen or the one or more immunogenic epitopes thereof. In some embodiments, the targeting sequence may be a proteosomal targeting sequence, for example a ubiquitin sequence. In some embodiments, the targeting sequence may be an endosomal targeting sequence.

In another aspect, there is provided a dendritic cell that is derived from a pluripotent stem cell, the pluripotent stem cell stably modified with a nucleic acid molecule encoding an antigen or one or more immunogenic epitopes thereof, wherein the dendritic cell expresses and presents the antigen or the one or more immunogenic epitopes thereof

The dendritic cell may be produced according to a method of the invention.

The dendritic cell may express one or more of CD11c, CD86 and HLA markers.

In another aspect, there is provided a vaccine comprising the dendritic cell of the invention.

The vaccine may further comprise an adjuvant and/or a pharmaceutically acceptable excipient or diluent.

In another aspect, there is provided a method of inducing an immune response in a subject, the method comprising: administering the dendritic cell or the vaccine of the invention, to a subject in need of immunity to the antigen.

The immune response may be a T-cell mediated immune response, including a CD8+ or a CD4+ T cell mediated response.

In the method, the dendritic cell may be autologous with the subject, or may be allogeneic with the subject. In some embodiments, the dendritic cell may at least partially MHC-matched with the subject.

The subject may be a subject is in need of treatment for cancer, and the antigen may be a tumour antigen. For example, the subject may be in need of treatment of melanoma, colorectal cancer, glioma, prostate cancer, breast cancer, ovarian cancer, lung cancer, pancreatic cancer, or gastrointestinal cancer.

In another aspect, there is provided use of the dendritic cell or the vaccine of the invention for inducing an immune response in a subject, or in the manufacture of a vaccine for inducing an immune response in a subject.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

The figures, which illustrate, by way of example only, embodiments of the present invention, are as follows.

FIG. 1: Schematic summary of the antigen-loading strategy for DC vaccine production from human pluripotent stem cells (hPSCs). (Above dotted line) In a traditional patient blood cell-dependent platform, antigen-loading is limited to DCs, wherein antigen payloads in various forms are delivered into DCs by conventional antigen-loading approaches. (Below dotted line) In the hPSC-DC platform, antigen-loading can be done in hPSCs by antigenically modifying the hPSCs. From such antigenically modified hPSCs, antigen-loaded DCs can be generated without the need for a conventional antigen-loading step. Using this antigen-loading strategy, there are no more requirements of clinical-grade payload production and additional DC manipulation. Thus, DC vaccine production from hPSCs is significantly simplified.

FIG. 2: Tumor antigen gene-modified hPSCs produce tumor antigen-expressing DCs. (A): Structure of lentivector LV.MP carrying a tumor antigen gene MART-1. (B): GPF expression in H1.MP cells, a H1 cell line generated by LV.MP transduction and G418 selection, as detected by flow cytometry. (C): MART-1 expression in H1.MP cells as measured by RT-PCR. (D): MART-1 expression in H1.MP cells as measured by immunostaining. (E): GFP expression in H1.MP-derived DCs (H1.MP-DCs) as detected by flow cytometry. (F): MART-1 expression in H1.MP-DCs as measured by RT-PCR.

FIG. 3: DCs derived from tumor antigen gene-modified hPSCs present tumor antigen. (A): Proliferation of GFP^{high} H1.MP cells after sorting. (B): GFP expression in sorted GFP^{high} H1.MP cells as detected by flow cytometry. (C): MART-1 expression in GFP^{high} H1.MP cells as measured by RT-PCR. (D): MART-1 expression in GFP^{high} H1.MP cells as measured by immunostaining. (E): Expansion of primed MART-1-specific CD8+ T cells by GFP^{high} H1.MP-derived DCs as detected by pentamer staining and flow cytometry (priming/restimulation: no DC/no DC (top left panel); MART1 peptide-pulsed H1-DC/H1-DC (top right panel); MART1 peptide-pulsed H1-DC/GFP^{high} H1.MP-DC (bottom left panel); GFP^{high} H1.MP-DC/GFP^{high} H1.MP-DC (bottom right panel).

FIG. 4: Modification of hPSCs with tumor antigen epitope-coding minigene. (A): Structure of lentivector LV.ME carrying MART-1 epitope-coding minigene. (B): GPF expression in H1.ME cells, a H1 cell line generated by LV.ME transduction and G418 selection, as detected by flow cytometry. (C): MART-1 expression in H1.ME cells as measured by RT-PCR. (D): SSEA-4 expression in H1.ME as detected by immunostaining.

FIG. 5: Tumor antigen epitope-coding minigene is expressed in DCs derived from minigene-modified hPSCs. (A): Morphology of DCs derived from minigene-modified hPSCs (H1.ME-DCs). (B): Yield of H1.ME-DCs. The statistical significance of difference was determined by two-sided Student's t-test (mean±SD, n=10). (C): Phenotype of H1.ME-DCs. (D): GFP expression in H1.ME-DCs as detected by flow cytometry. (E): Expression of MART-1 epitope-coding minigene in H1.ME-DCs as measured by RT-PCR. (F): CD83 expression on H1.ME-DCs after treatment with TNF. (G): Allostimulatory function of H1.ME-DCs on CD4+ T cells after treatment with TNF. The percentages of divided CD4+ T cells are indicated.

FIG. 6: DCs derived from minigene-modified hPSCs efficiently prime tumor antigen-specific T cell response. (A): Induction of MART-1-specific CD8+ T cell response by H1-DCs pulsed with MART-1 peptide at concentrations of 0, 1, 5, 10 and 20 μg/ml. (B-C): Induction of MART-1-specific CD8+ T cell response by H1.ME-DCs in PBLs of low responsiveness. The antigen-specific T cells were stained by pentamer and detected by flow cytometry nine days after DC:PBL coculture. (B): Contour plots of representative experiment. The numbers in plots indicate the percentage of pentamer+CD8+ cells in total T cells. (C): Quantitative analysis of the experiments. The statistical significance of differences were determined by two-sided Student's t-test (mean±SD, n=6). (D-E): Induction of MART-1-specific CD8+ T cell response by H1.ME-DCs in PBLs of high responsiveness. (F): Comparing T cell priming ability of H1.ME-DCs and MART-1 peptide-pulsed moDCs. The statistical significance of differences were determined by two-sided Student's t-test (mean±SD, n=5). (G): Comparing T cell priming ability of MART-1 peptide-pulsed H1-DCs and H1.ME-DCs after prolonged culture. H1-DCs were pulsed with MART-1 peptide, washed and further cultured for seven days before applying for priming. Unpulsed H1-DCs and H1.ME-DCs were employed as controls. (H): Induction of MART-1-specific CD8+ T cell response by H1.ME-DCs using different DC:PBL ratio (0, 1:10, 1:7.5, 1:5 and 1:2.5 DC:PBL).

FIG. 7: CTLs expanded by DCs derived from minigene-modified hPSCs are immunocompetent. (A): Expansion of MART-1-specific CD8+ T cells by H1.ME-DCs in bulk culture. HLA-A2+ PBLs were primed and then restimulated twice with H1.ME-DCs. MART-1-specific T cell expansion during this process was monitored by flow cytometry at the indicated time points. The percentages of pentamer+CD8+ cells in total T cells are shown in the representative contour plots. (B): Phenotype of MART-1-specific T cells expanded by H1.ME-DCs. (C): GrB secretion by MART-1-specific T cells expanded by H1.ME-DCs as measured by ELISPOT. The statistical significance of differences were determined by two-sided Student's t-test (mean±SD, n=3). (D): Specific cytotoxicity of MART-1-specific T cells expanded by H1.ME-DCs. The statistical significance of differences were determined by two-sided Student's t-test (mean±SD, n=3, *p<0.002).

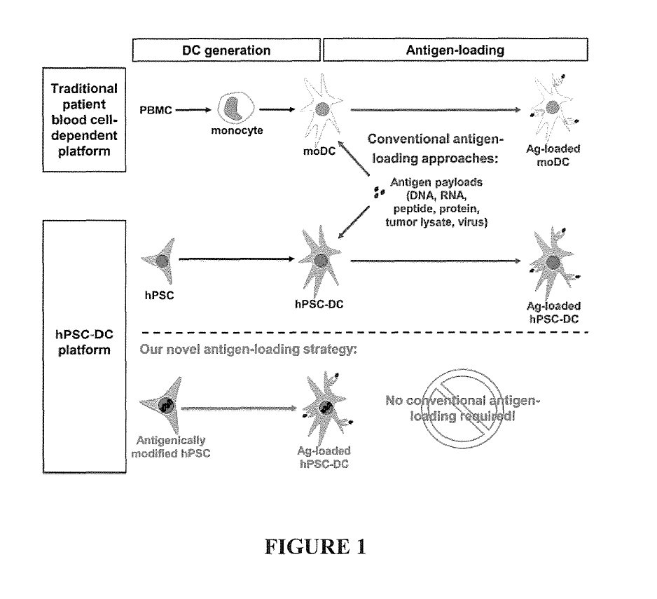

### DETAILED DESCRIPTION

Dendritic cells (DCs) were first discovered in 1973 by Prof Ralph M. Steinman, who was awarded the Nobel Prize in Physiology or Medicine in 2011. DC vaccines have been widely tested in clinical trials for cancer immunotherapy; Dendreon's Provenge™ is the first ever FDA-approved DC-based vaccine for prostate cancer.

In contrast to the conventional methods for antigen loading of DCs, the methods as described herein provide a simpler antigen-loading solution that allows for production of DC vaccine from pluripotent stem cells (PSCs), including human PSCs (hPSCs), which have been modified with antigen genes, including tumor antigen genes. Such antigenically modified PSCs are able to differentiate into functional antigen-presenting DCs.

Specifically, PSCs are stably modified using antigen genes, including in the form of a full-length antigen gene or an artificial antigen epitope-coding minigene.

Such genetically antigenically modified PSCs are able to differentiate into antigen-presenting DCs that may be used to prime an antigen-specific T cell response and further expand these specific T cells during restimulation processes. The expanded antigen-specific T cells may be potent antigen-specific effectors with central memory and effector memory phenotypes.

Thus, immunocompetent antigen-loaded DCs can be directly generated from antigenically modified PSCs using the methods of the invention. Using such strategy, the conventional antigen loading process that is done in a differentiated DC can be eliminated, thus significantly simplifying the DC vaccine production.

This method is applicable for a variety of different antigen types, including tumor, bacterial, viral and autoimmune disease antigens, using antigen genes in form of both full-length sequence and a minigene encoding repeats of an epitope selected from the full-length sequence. The polypeptide products of these antigen genes can be processed and presented by the derived DCs, which may then efficiently induce an antigen-specific CD8+ or CD4+ T cell response.

Thus, immunocompetent antigen-presenting DCs can be directly generated from antigenically modified PSCs, thereby eliminating the requirements of antigen payload production and extra DC manipulation to deliver the payload, in contrast to previous techniques relating to antigen loading of DCs.

This novel antigen loading strategy may also enhance DC vaccine efficacy. In terms of antigen presentation pathway, the antigens are synthesized endogenously from the transgene introduced into the precursor PSC, and thus the expressed antigen or epitope may be naturally channeled to the endogenous pathway for presentation by MHC class I, which is the preferred pathway for a tumor antigen presentation by DCs for use in a cancer vaccine.

In addition to MHC class I epitopes, MHC class II-restricted epitopes may be presented by the DCs for use in a vaccine. It is well-known that CD4+ helper T cells also contribute to anti-tumor immunity by activating DCs and by producing optimal cytokines [27]. DC vaccines that activate CD4+ helper T cells simultaneously may be useful to further improve tumor antigen-specific CTL response. By using a transgene that includes HLA class II-restricted epitopes, this antigen-loading strategy may also be applied for presenting antigens to CD4+ T cells.

With respect to antigen presentation duration, constitutive expression of the antigen may be used to provide a continuous supply of antigens from the transgene expression, which may prolong antigen presentation by the derived DCs, thus improving DC immunogenicity.

From the viewpoint of DC vaccine development, a batch of antigenically modified PSCs may be expanded and thus may provide an unlimited amount of standardized antigen-loaded DCs. Thus, the process may be useful for optimizing other aspects of DC vaccines due to the stable supply of standardized DCs.

Thus, there is provided a method of producing an antigen-loaded dendritic cell.

The method involves modification of a pluripotent stem cell (PSC) with a nucleic acid molecule that encodes an antigen that is to be used to elicit an immune response, or that encodes one or more immunogenic epitopes of such an antigen.

As used herein, reference to a “cell”, including when used in context of a pluripotent stem cell or a dendritic cell, is intended to refer to a single cell as well as a plurality of cells or a population of cells, where context allows, unless otherwise specified. Similarly, the term “cells” or “population” of cells is also intended to refer to a single cell, where context allows, unless otherwise specified.

The cell may be an in vitro cell, may be grown in batch culture or in tissue culture plates, may be in suspension or may be attached to culture support surface. The cell may be formulated into a vaccine, and may be administered to a subject and thus may be found in an in vivo context.

The pluripotent stem cell used in the method may be any pluripotent stem cell. A pluripotent stem cell is any undifferentiated stem cell that has the potential to differentiate into any type of a cell in the organism from which the stem cell originates. For example, a pluripotent stem cell can differentiate into a cell from one of the three germ layers, the endoderm, ectoderm or mesoderm, or any cell type arising from the endoderm, ectoderm or mesoderm, including partially differentiated or fully differentiated cell types. A pluripotent cell may be identified by its expression of a pluripotency marker, for example expression of one or more of OCT4, TRA-1-60, SSEA-4, SOX2, KLF4, c-MYC, REX1, NONOG, LIN28 and DNMT3B.

The pluripotent stem cell may be an embryonic stem cell (ESC), including for example an embryonic stem cell from an established cell line, including commercially available cell clines. The embryonic stem cell may be derived by somatic cell nuclear transfer, i.e. an ntESC, or may be derived from an unfertilized egg by parthenogenesis, i.e. a pESC.

The pluripotent stem cell may be an induced pluripotent stem cell (iPSC). As used herein, an iPSC is a pluripotent stem cell that has been induced to a pluripotent state from a non-pluripotent originator cell, for example a partially or fully differentiated cell that can be induced to become pluripotent by exposure to appropriate conditions and transcription factors or other protein factors that regulate gene expression profiles in pluripotent cells. The iPSC is thus a pluripotent cell that has been derived from a non-pluripotent originator cell and is not an embryonic stem cell. Methods for generating iPSCs from differentiated cells are known, including for example methods using Yamanaka factors, originally identified in 2006 by Professor Shinya Yamanaka, including as described in Takahashi and Yamanaka (2006) Cell 126:663-676.

The PSC may be from any animal, including a mammal, including a non-human mammal or a human. In some embodiments, the PSC used is a human PSC (hPSC).

The PSC may be from an established cell line, for example an ESC line or an iPSC line that is commercially available.

If the DCs are to be used for treatment, for example in a vaccine, the PSC may be from the same species to which the resulting antigen-loaded DC is to be administered, and thus may be allogenic with the intended subject for treatment. The PSC may be partially MHC-matched or fully MHC-matched with the intended subject for treatment. The PSC may be derived from cells from the subject to which the resulting antigen-loaded DC is to be administered, and thus may be autologous with the intended subject for treatment. Alternatively, the PSC may be derived from a person that is genetically related to the subject, or from a healthy donor that may not be genetically related to the subject.

The PSC used in the method is modified with a nucleic acid molecule that encodes an antigen or one or more immunogenic epitope of an antigen that is to be presented by the resulting DCs.

The antigen may be any antigen that can be encoded by a nucleic acid and which is desired to be expressed and presented by the DCs, which antigen-presenting DCs may be used as a vaccine. The antigen may be a full-length antigen that has a proteinaceous component, such as a protein or peptide. The full-length antigen may be an antigen that is further post-translationally modified upon expression in the DCs, for example a glycoprotein or a lipoprotein.

The antigen may be a tumor antigen, for example a protein or peptide expressed by tumor cells that is not typically expressed in a healthy, non-cancerous cell of the same cell lineage as the tumor cell. In some embodiments, the tumor antigen may be WT1, MUC1, EGFRvIII, HER-2, MAGE-A3, NY-ESO-1, PSMA, GD2, or MART1.

The antigen may be a viral antigen, for example a protein or peptide that forms part of a virus or that is expressed in a cell infected by the virus under control of the viral expression machinery. In some embodiments, the viral antigen may be EBV LMP2, HPV E6 E7, Adenovirus 5 Hexon, or HCMV pp65.

The antigen may be a bacterial antigen, including for example a protein or peptide expressed by a bacterium. In some embodiments, the bacterial antigen may be Mycobacterium bovis antigen.

The antigen may be disease-related antigen, including an autoimmune-related antigen, for example an antigen involved in or over expressed in an autoimmune disease or disorder. In some embodiments, the autoimmune-related antigen may be ppIAPP, IGRP, GAD65, or Myelin basic protein antigen.

Additionally or alternatively, one or more immunogenic epitopes may be encoded by the nucleic acid molecule. As used herein, an immunogenic epitope (also referred to as an epitope) is a portion of an antigen that is presented and recognized by T cell receptor, for example an epitope of an antigen as defined herein. An immunogenic epitope may be in the form of a linear sequence of amino acids that may be 5, 6, 7, 8, 9, 10, 11, 12, 13, 14, 15, 16, 17, 18, 19 or 20 amino acids in length.

As with the antigen, each of the one or more immunogenic epitopes has a proteinaceous portion that is encoded by the nucleic acid, and may be further post-translationally modified upon expression in the DCs.

In various embodiments, one, two, three, four, five, six, seven, eight, nine or ten immunogenic epitopes may be encoded by the nucleic acid molecule.

Each of more than one immunogenic epitopes may be the same, or may be different epitopes. Thus, if more than one immunogenic epitope is encoded by the nucleic acid molecule, all of the immunogenic epitopes may have the same amino acid sequence, some may have the same amino acid sequence and some may have a different amino acid sequence, or each may have a different amino acid sequence. In order to improve the T cell response to the epitope, in some embodiments, more than one immunogenic epitope is encoded by the nucleic acid molecule and each of the more than one immunogenic epitopes has the same amino acid sequence.

If more than one immunogenic epitope is encoded by the nucleic acid molecule, each immunogenic epitope may be encoded within a different open reading frame, or may be encoded within the same open reading frame. When encoded in the same open reading frame, each of the immunogenic epitopes may be separated by a spacer sequence of amino acids. For example, each immunogenic epitope may be separated by from 1 to 20 amino acids in a protein sequence encoded by the nucleic acid molecule.

The nucleic acid molecule may be any nucleic acid molecule that comprises a coding sequence for the antigen or one or more immunogenic epitopes and that may be transferred into a PSC for expression of the sequences encoding the antigen or one or more immunogenic epitopes. In some embodiments, the nucleic acid molecule is DNA.

The nucleic acid molecule may be any type of nucleic acid molecule that can be stably maintained in a PSC and a DC. For example, the nucleic acid molecule may be an extrachromosomal vector that is replicated and divided so as to be stably maintained even in an expanding cell population. Or in another example, the nucleic acid molecule may be inserted into a chromosome within the host PSC and thus chromosomally integrated into the PSC.

Thus, the PSC may be stably modified with the nucleic acid molecule.

Thus, in some embodiments, the nucleic acid molecule is a retroviral vector, including a retroviral vector that can stably integrate into the genome of the PSC into which it is introduced. Retroviral vectors include, for example, MMLV vectors, or lentiviral vectors. In some embodiments, the nucleic acid molecule is a lentiviral vector.

As will be appreciated, a suitable promoter will be operably linked to the coding region for the antigen or one or more immunogenic epitopes to allow for expression in a DC, and which in some embodiments may be selected to also allow for expression in a PSC. A coding sequence is operably linked to a promoter if the promoter activates the transcription of the coding sequence.

The promoter may thus be cell-type specific for dendritic cells or cells derived from peripheral blood lymphocytes or hematopoietic progenitor cells. The promoter may be a ubiquitous promoter that is expressed in PSCs and DCs. The promoter may be a constitutive promoter, for example a constitutive promoter active in DCs, or it may be an inducible promoter including any necessary encoded elements such as an operator required for induction of expression from the inducible promoter.

The nucleic acid molecule may also include other sequences which may be operably linked to the coding sequence, or which may be incorporated into the coding sequence open reading frame.

For example, a proteasomal targeting sequence may be included in order to direct the expressed protein product to the MHC I antigen degradation pathway and thus for inclusion for antigen presentation by an MHC I molecule in the DC. Proteasomal targeting sequences are known, and include for example, a ubiquitin sequence. The proteasomal targeting sequence may be included in the open reading frame so that it is fused to the proteinaceous portion of the antigen or one or more immunogenic epitopes when expressed from the nucleic acid molecule.

In another example, an endosomal targeting sequence or sorting signal may be included in order to direct the expressed protein towards the endosomal pathway for antigen presentation by an MHC II molecule in the DC. Such endosomal targeting sequences or sorting signals are known. The endosomal targeting sequence or sorting signal may be included in the open reading frame so that it is fused to the proteinaceous portion of the antigen or one or more immunogenic epitopes when expressed from the nucleic acid molecule.

In the methods, the PSC is modified with the nucleic acid molecule. Modification of the PSC refers to introducing the nucleic acid molecule into the cell using molecular cloning and recombinant techniques. Such techniques are known in the art, including techniques involving transfection, transduction or transformation of the cell with the nucleic acid molecule such that the nucleic acid molecule is taken up by the cell.

The modification of the PSC may be performed using a nucleic acid molecule and methodology that results in stable modification of the PSC such that the PSC maintains the nucleic acid molecule while cultured in an undifferentiated state, during the differentiation to a DC and the DC maintains the nucleic acid molecule upon culturing after differentiation, thus allowing for long term expression of the antigen or one or more immunogenic epitopes by the DC. For example, the differentiated DC that contains the nucleic acid may express the antigen or one or more immunogenic epitopes upon culturing for 7 days or longer, for 2 weeks or longer, for 3 weeks or longer, or for 4 weeks or longer.

In some embodiments, stable modification involves integration of the nucleic acid molecule into the genome of the modified PSC. For example, if a retroviral vector is used as the nucleic acid molecule, including for example a lentiviral vector, the retroviral vector may stably integrate into the cellular DNA of the modified PSC, and cells that arise upon proliferation or differentiation of the modified PSC will also include the nucleic acid molecule inserted into the cellular DNA.

It may be desirable to obtain an enriched cell population of modified PSCs. Thus, following modification of PSC with the nucleic acid molecule, the cells may be sorted to select for cells that have been modified with the nucleic acid molecule, using cell sorting techniques. Cell sorting techniques are known in the art. In this case, the nucleic acid molecule may include an expression construct that expresses a marker that is detectable using cell sorting methods to identify modified PSCs and to select such modified PSCs by the sorting method. For example, the marker may be a fluorescent protein that is expressed within the PSCs, even in an undifferentiated state. For example, the marker may be under the control of the EF1alpha promoter, which can be expressed in PSCs.

The PSC that has been modified with the nucleic acid molecule is then induced to differentiate into a dendritic cell. Inducing differentiation as used herein refers to providing suitable growth conditions, including a culture medium containing appropriate growth factors and nutrients, at a temperature and for a time necessary for the PSC to differentiate into a DC.

Differentiation methods to induce PSCs to become dendritic cells are known in the art and have been previously described [9, 10, 12, 31]. For example, the PSCs may be co-cultured with feeder cells to derive myeloid progenitors, which are then expanded and further differentiated into dendritic cells.

The differentiated DC is able to express and present the antigen or the one or more immunogenic epitopes from the nucleic acid molecule. Thus, once differentiated, in order to express the antigen, the DC is cultured under conditions that allow for antigen expression from the nucleic acid, including in the presence of any transcription factors or regulatory factors that may be required to regulate expression of the coding sequence encoding the antigen or the one or more immunogenic epitopes. Expression of the antigen may be under control of a promoter that is constitutively active in DCs, which may facilitate antigen expression upon administration of the DCs to a subject for treatment. However, in some embodiments, the coding sequence for the antigen or the one or more immunogenic epitopes may be under the control of an inducible promoter. In such case, any factor or condition required to induce expression from the promoter is also included in the culture conditions.

Once the antigen or one or more immunogenic epitopes are expressed within the DC, the antigen or immunogenic epitope is thus presented by the DC.

MHC I antigen presentation by an antigen presenting cell, including a DC, involves internal proteolytic digestion of the antigen by the proteasome into peptide fragments, and transport of the fragments to the endoplasmic reticulum where the peptides are loaded into a peptide loading complex that contains an MHC I molecule. The MHC I molecule will recognize and bind a fragment, and the MHC I/peptide complex is then transported to the external surface of the cell membrane, which allows for the MHC I/peptide complex to be recognized by and to activate the appropriate CD8+ T cell population.

In addition to MHC I antigens and epitopes, MHC class II antigens and epitopes may be used. Once expressed within a cell, the cytosolic antigen may be sorted to the endosome by an endosomal sorting signal, followed by degradation of the antigen, and recognition and binding by an MHC II molecule. The MHC II/peptide complex is then transported to the external surface of the cell membrane, which allows for the MHC II/peptide complex to be recognized by and to activate the appropriate CD4+ T cell population.

If desired, the DCs may be further matured by culturing in the presence of a cytokine, for example tumor necrosis factor (TNF) or another maturation cocktail, for example lipopolysaccharide (LPS) together with interferon gamma (IFN-γ), or other maturation reagents, such as for example agonists of Toll-like receptor (TLR agonists). Maturation of the DCs prior to administration to a subject may improve the ability of the DCs to prime or restimulate the appropriate T cell response upon administration to a subject.

Thus, the methods yield a DC that is genetically modified to result in expression and presentation of the desired antigen or epitope. For vaccines based on DCs, antigen loading of the DCs is one of the most crucial steps, and effectively defines the specificity of anti-tumor immune responses elicited by the DC vaccine. The methods as described herein use genetic modification of a pluripotent stem cell, which is then differentiated into a dendritic cell. The use of genetic modification of a pluripotent stem cell followed by differentiation can result in a DC population that stably expresses the desired antigen, which expression can be maintained over a relatively long culture period, for example, 7 days, or even longer.

This method of producing the DC thus negates the need for peptide-pulsing, protein-loading, tumor lysate-loading, RNA/DNA transfection or viral transduction, which are commonly used techniques previously described [11]. Avoidance of the previously known antigen loading methods also avoids additional cell manipulations. The use of pluripotent stem cells to derive the DC population may provide a consistent cell source with sufficient numbers of cells to allow for large-scale DC vaccine production, thus avoiding batch-to-batch inconsistencies seen with small batch vaccine production.

Thus, the method uses a genetically modified PSC to produce a DC that presents the desired antigen or epitope.

Accordingly, there is also provided a dendritic cell derived from a pluripotent stein cell that has been stably modified with a nucleic acid molecule encoding an antigen or one or more immunogenic epitopes thereof.

The DC is thus able to express and present the antigen or the one or more immunogenic epitopes.

The DC may thus be identified by presentation of the antigen or the one or more immunogenic epitopes at the cell surface when cultured under conditions that result in expression of the antigen or the one or more immunogenic epitopes. Antigen presentation on the DCs may be confirmed, for example by testing the ability of the DCs to stimulate the antigen-specific T cell response.

As well, the DC expresses DC-specific cell markers, which may include for example, one or more of CD11c, CD40, CD83, CD86 and HLA-DR.

Due to presentation of the antigen or the one or more immunogenic epitopes, the DC is able to induce a T cell response. That is, the DC is able to prime an antigen- or epitope specific response in a T cell population, or is able to restimulate a T cell population that has previously been primed with the specific antigen or epitope. Thus, it is possible to test a T cell population by incubating the antigen-presenting DC with the T cell population and detecting whether the T cell population becomes primed, restimulated or expanded in response. As well, a T cell population that has been exposed to the antigen or epitope may be tested for response using a labelled epitope.

As indicated above, the T cell population that is primed or restimulated may be a CD8+ or a CD4+ T cell population, depending on whether the antigen is presented by an MHC I or an MHC II molecule, respectively.

The DC may be produced in accordance with the methods as described herein.

The DC may be contained within a population of cells, and thus there is also provided a population or plurality of cells comprising the DCs.

In the population of cells, the majority or all of the cells may be DCs that present the antigen or one or more immunogenic epitopes. For example, the proportion of genetically modified DCs that present the antigen or one or more immunogenic epitopes present in the population of cells may be at least 50%, at least 60%, at least 70%, at least 75%, at least 80%, at least 85%, at least 90%, at least 95%, at least 96%, at least 97%, at least 98%, or at least 99%. In some embodiments, the proportion of genetically modified DCs that present the antigen or one or more immunogenic epitopes may be from about 50% to about 75%, or about 55% to about 60%.

The population contains DCs that originated from PSCs. Thus, the population may also contain non-differentiated PSCs, partially differentiated PSCs, and even some transdifferentiated cells, although in some embodiments the large majority of cells, or even all of the cells, will be DCs.

The population may be enriched for DCs that present the antigen or one or more immunogenic epitopes present in the population of cells, for example by using cell sorting techniques, in order to increase the proportions of cells in the population that are DCs that present the antigen or one or more immunogenic epitopes present in the population of cells.

The use of PSCs, including hPSCs, in the methods as described herein to derive DCs that are modified with a nucleic acid molecule encoding a desired antigen or one or more immunogenic epitopes thereof may yield a consistent and renewable cell source for vaccine production. Thus, the described methods yield antigenically modified DCs that may allow for centralized and large-scale DC vaccine production, as well as individually tailored DC vaccines when the iPSC is derived from a subject that is to be treated with the vaccine. The herein described methods of preparing the antigenically modified DCs by genetically modifying precursor hPSCs that are differentiated into antigen-presenting DCs avoids any conventional antigen loading step, thus simplifying the production process.

Thus, genetically modified DCs such as those derived from the described methods may be used to prime and expand an antigen-specific T cell response, or restimulate and expand an antigen-specific T cell response to the antigen or epitope presented by the DCs. Such expanded antigen-specific T cells may act as immunocompetent antigen-specific effectors with central memory or effector memory phenotypes, and thus may confer an immune response against the antigen or epitope to an individual when the DCs are administered as a vaccine. Accordingly, the DC, including when contained in a population or plurality of cells, may be formulated as a vaccine for administration to a subject.

Thus, there is also provided a vaccine comprising a dendritic cell as described herein.

The concentration of DCs included in the vaccine is chosen in order to provide a dose containing an effective amount of DCs. As used herein, the term “effective amount” as used herein means an amount effective, at dosages and for periods of time necessary to achieve the desired result, for example to the amount necessary to prime or boost an immune response to the antigen or epitope in the subject.

For example, the vaccine may be formulated to provide a dose of from about 1×10⁵ to about 1×10⁹ of the DCs, or about 1×10⁶ to about 1×10⁸ of the DCs, or about 1×10⁶ to about 5×10⁷ of the DCs.

In some embodiments, the initial, priming dose of the vaccine may contain a higher count of the DCs than subsequent boosting, restimulation doses.

In the vaccine, the DCs are formulated live in a solution.

The solution may thus contain pharmaceutically acceptable concentrations of salt, buffering agents, preservatives and various compatible carriers, which may assist in maintaining the live cells in the formulation. The solution which contains the cells may therefore be designed to be isotonic with the cells, and may also be pH buffered. Thus, when formulated within a vaccine, the carrier solution may be designed so as to prevent, minimize or reduce cell lysis prior to administration of the vaccine to a subject.

If the vaccine is to be stored frozen, the vaccine may include a cryoprotectant, for example DMSO.

The vaccine may further include an adjuvant if desired, to assist in induction or re-stimulation of an immune response, including to prolong or enhance the immune response. Suitable adjuvants are known in the art, including adjuvants that enhance a T cell response. For example, the adjuvant may comprise Alum adjuvant, Freunds adjuvant, a muramyl peptide, cyclophosphamide, ISCOMS, MAPS, thymosin alpha 1, levamisole, isoprinosine or TLR ligands.

The proportion and identity of the various ingredients included in the solution is determined by chosen route of administration, compatibility with live cells, and standard pharmaceutical practice. Generally, the vaccine will be formulated with components that will not kill or significantly impair the biological properties of the DCs.

A person skilled in the art would know how to prepare suitable vaccine formulations. Conventional procedures and ingredients for the selection and preparation of suitable vaccines and live cell preparations are described, for example, in Remington's Pharmaceutical Sciences and in The United States Pharmacopeia: The National Formulary.

The DCs and the vaccine can thus be used to effect an immune response, including priming an initial response or restimulating or boosting a response in already primed T cells.

Thus, there provided a method of inducing an immune response. The immune response is effected by contacting the DCs, including when formulated as the vaccine, with a T cell.

The T cell may be an in vitro T cell, including a CD4+ T cell or a CD8+ T cell, and thus the DCs and /or vaccine may be used in an in vitro method to activate, prime or restimulate an in vitro population of T cells.

The DC, including when formulated as the vaccine, may also be used in vivo to elicit an immune response in a subject, including a T-cell mediated immune response as described herein.

Thus, the DC or vaccine may be administered to a subject in whom an immune response against the antigen or one or more immunogenic epitopes is desired to be raised. In some embodiments, the vaccine comprising the DCs is administered to the subject.

The immune response may be a T cell mediated immune response, meaning that the antigen or one or more immunogenic epitopes presented on the surface of the DC is able to be recognized by a T cell and is able to induce a response in the T cell, such as causing the T cell to expand to provide an antigen-specific T cell population. The T cell mediated immune response may be a primary response, in which the T cell has not be previously exposed to the antigen or epitope, or it may be a restimulation of a T cell that has been previously exposed to the antigen or epitope or which is a cell in an expanded population expanded from a T cell that has been previously exposed to the antigen or epitope. The T cell may be a CD8+ T cell or may be a CD4+ T cell.

The subject may be any animal, including a mammal, including a non-human mammal or a human, in whom an immune response to the antigen or one or more immunogenic epitopes is desired to be induced, or who is in need of immunity to the antigen or one or more immunogenic epitopes. In some embodiments, the subject is a human.

The subject may be in need of immunity against a pathogen, including a viral or bacterial pathogen. The subject may be in need of treatment for a disease in which the disease may be treated by immunotherapy, including for example cancer. The cancer may be, for example, melanoma, colorectal cancer, glioma, prostate cancer, breast cancer, ovarian cancer, lung cancer, pancreatic cancer, or gastrointestinal cancer.

The subject may have been previously exposed to the antigen or the one or more immunogenic epitopes thereof. For example, the subject may have previously been vaccinated against a pathogen or may have come into contact with a pathogen from which the antigen or one or more immunogenic epitopes are derived. The subject may have a disease associated with expression of the antigen.

In other embodiments, the subject may not have been previously exposed to the antigen prior to administration of the vaccine.

The DC or vaccine may be administered by injection, including for example intravenously, subcutaneously, intradermally, or intranodally. If the DCs express a tumor antigen, it may be desirable to select an injection site remote from the tumor in order to avoid lymph nodes located near the tumor, which may be influenced by tumor-derived immunosuppression factors and thus which may drain away the administered DCs.

An effective amount of the vaccine is administered to the subject in order to induce or elicit the desired immune response as indicated herein, including priming of an initial response or restimulation of previously stimulated response.

The concentration and amount of the vaccine and the number and timing of doses to be administered will vary, depending on a variety of factors, including the identity of the antigen or one or more immunogenic epitopes, the type of immune response to be elicited, whether the vaccine is to be administered to protect against pathogen infection or in treatment of a disease or disorder, the duration of treatment, as well as the mode of administration, the age and health of the subject, the nature of concurrent therapy (if any), the specific route of administration and other similar factors. These factors are known to those of skill in the art and can be addressed with minimal routine experimentation.

The vaccine may be administered in one or more doses. For example, the DC or vaccine may be administered as an initial priming dose, followed by one or more boost doses, or as one or more boost doses, at suitable intervals.

The tuning and size of subsequent boost doses may vary, depending on the ability of the antigen to prime and/or restimulate a T cell response. For example, tumor antigens may require more frequent boosting schedule depending on the strength of the elicited T cell response.

The vaccine may be administered in combination with other treatments. For example, the vaccine may be administered in combination with a traditional vaccine derived from an attenuated or killed pathogen or a lysate or component of a pathogen. The vaccine may be administered in combination with treatment for a disease, such as any disease that may benefit from treatment with immunotherapy, including for example cancer.

If administered in combination with another treatment, the vaccine may be administered simultaneously with the other treatment, including formulated together with a medicament for the other treatment or formulated separately.

The vaccine and other treatment may be administered with overlapping timing, meaning that at least a portion of the time period of treatment with the vaccine coincides with at least a portion of the time period of treatment with the other treatment. The vaccine may be administered sequentially with the other treatment, including in a time period prior to the time period of the other treatment or in a time period subsequent to the time period of the other treatment.

As described above, the DCs included in the vaccine may be allogenic with the subject. Thus, the DCs may be derived from the same species as thus subject, and may be partially MHC-matched or fully MHC-matched with the subject. The DCs may be derived from a PSC from a person that is genetically related to the subject, or from a healthy donor that may not be genetically related to the subject.

Unlike in the setting of regenerative medicine, wherein HLA-matched transplant is required for long-term engraftment, the requirement of histocompatibility in DC-based therapy is less stringent since long-term survival of DCs is not necessary. However, in such allogeneic setting, the DC should be chosen to induce and immune response before elimination by allo-reactive cytotoxic lymphocytes of the subject.

The DCs included in the vaccine may be autologous with the subject, and thus may be derived from PSCs from the subject.

Also contemplated are uses of the described DC and vaccine, in keeping with the methods as described herein, including use of the DC or vaccine for inducing an immune response in a subject or in the manufacture of a medicament for inducing an immune response in a subject. As well, the described DC or vaccine may be for the uses as described herein, including for use in the induction of an immune response in a subject.

The described methods, dendritic cells, vaccines and uses are further exemplified by the following non-limiting examples.

### EXAMPLES

Materials and Methods

Cell Culture and DC Generation

A hPSC line, H1 (WiCell Research Institute, Madison, Wis.), was maintained with a serum-free and feeder-free culture system using mTeSR1 medium (StemCell Technologies, Vancouver, BC, Canada) and Matrigel (BD Biosciences, San Diego, Calif.)-coated six-well plates according to manufacturer's technical manual. OP9 cells (American Type Culture Collection [ATTC], Manassas, Va.) were cultured with α-MEM (Life Technologies, Carlsbad, Calif.) supplemented with 20% fetal bovine serum (FBS) (HyClone, Logan, Utah). T2 cells (ATCC) were cultured with IMDM (Life Technologies) supplemented with 20% FBS.

To derive human DCs from H1 cells, we used a three-step protocol as described previously [9, 10, 12]. In brief, OP9 cells were seeded on 0.1% gelatin (Sigma-Aldrich, St Louis, MO) -coated T75 flask. Upon confluence, the culture was fed by changing half of the medium, and then was overgrown for 4-6 days. 1-1.5×10⁶ H1 cells were then seeded and differentiated on the overgrown OP9 cells in α-MEM supplemented with 10% FBS and 100 μM monothioglycerol (Sigma-Aldrich). The coculture was fed on day 4 and 6 by changing half of the medium and was harvested on day 9 using 1 mg/ml collagenase IV (Life Technologies) and 0.05% trypsin-0.5 mM EDTA (Life Technologies). The harvested cells were further cultured for 10 days in a poly 2-hydroxyethyl methacrylate (Sigma-Aldrich)-coated T75 flask using α-MEM supplemented with 10% FBS, 100 μM monothioglycerol and 100 ng/ml GM-CSF (Peprotech, Rocky Hill, N.J.). To generate human DCs, these cells were then purified by density gradient centrifugation using 25% Percoll solution (Sigma-Aldrich) and cultured in StemSpan serum-free expansion medium (StemCell Technologies) supplemented with 1% lipid mixture 1 (Sigma-Aldrich), 100 ng/ml GM-CSF and 100 ng/ml IL-4 (Peprotech) for 8-12 days.

To obtain human peripheral blood lymphocytes (PBLs), frozen HLA-A2+ PBMCs from healthy donors (StemCell Technologies) were thawed and cultured in complete RPMI 1640 medium, which contains RPMI 1640 (Life Technologies) supplemented with 10% heat-inactivated human serum AB (Gemini Bio-Products, West Sacramento, Calif.), 2 mML-glutamine (Life Technologies), 0.1 mM nonessential amino acids (Life Technologies), and 0.1 mM 2-mercaptoethanol (Life Technologies). After 2-hour incubation, the cells in suspension were harvested as PBLs. To derive moDCs, the plastic-adherent cells were differentiated in DC-differentiation medium for 6 days.

Lentivector Preparation and hPSC Modification

Two types of lentivectors were generated using two different transfer plasmids. To construct a transfer plasmid carrying a tumor antigen gene MART-I, the coding sequence of MART-1 was cloned from Plasmid MART-1 (ATCC) by PCR to include a Kozak sequence upstream of its start codon and EcoRI and BamHI restriction sites at its termini. These two sites were used to insert MART-1 gene into pCDH-EFI-MCS-IRES-coGFP-Neo (System Biosciences, Mountain View, Calif.). To construct another transfer plasmid carrying a gene encoding four repeats of a HLA-A2-restricted MART-1 epitope (MART-1₂₆₋₃₅A27L, ELAGIGILTV [SEQ ID NO: 1]), a minigene was synthesized (1st BASE, Singapore). This minigene encodes the following amino acid sequence:

[SEQ ID NO: 2]
MQIFVKTLTGKTITLEVEPSDTIENVKAKIQDKEGIPPDQQRLIFA
GKQLEGRTLSDYNIQKESTLHLVLRLRGVVNSEFKHEELAGIGILT
VAEFKSEELAGIGILTVAEEELAGIGILTVAEEELAGIGILTVAEE
VNRA

In the above sequence, a ubiquitin sequence (italic and underlined) was placed before the sequence of four MART-1 epitopes (bold and underlined) for proteasomal targeting and the codon usage was optimized for expression in human cells. The minigene was cloned and inserted into pCDH-EF1-MCS-IRES-coGFP-Neo using NheI and BamHI sites. Lentivectors, named LV.MP and LV.ME, were produced by contransfecting 293FT cells (Life Technologies) using the above-described constructs and packaging plasmids (System Biosciences). Virus titers were determined using 293FT by transduction with virus after serial dilution and subsequent antibiotic selection.

To genetically modify H1 cells, H1 cell clumps were seeded at a low cell density on Matrigel-coated six-well plates. Two days later, H1 cells were transduced by incubating with LV.MP or LV.ME at an MOI of 10 for 6 hours. Antibiotic selection with 50 μg/ml G4I 8 (Life Technologies) was started 3 days after transduction. The resulting G418-resistant H1 lines, designated as H1.MP or H1.ME, were used to derive DCs, designated as H1.MP-DCs or H1.ME-DCs for downstream experiments.

RT-PCR and Immunostaining

To detect MART-1 gene or minigene expression, total RNA of modified H1 cells or their DC progenies were extracted using TRIzol Reagent (Life Technologies). First-strand cDNA was then synthesized using SuperScript III First-Strand Synthesis System for RT-PCR (Life Technologies). 1 μl of cDNA reaction mix was used to amplify the whole MART-1 gene or the minigene using PCR SuperMix (Life Technologies). The PCR products were separated by electrophoresis in 1% agarose gel.

To detect MART-1 protein expression, the modified H1 cells were fixed with 4% paraformaldehyde (Sigma-Aldrich) and incubated with a primary antibody against MART-1 (Santa Cruz Biotechnology, Dallas, Tex.) for one hour. After washing, a secondary antibody goat anti-mouse IgG-TR (Santa Cruz Biotechnology) was used for visualization under a fluorescence microscope.

Priming, Expansion and Detection of Tumor Antigen-Specific T Cells

To prime a tumor antigen-specific T cell response, 1×10⁵ modified H1-derived DCs were matured using 20 ng/ml TNF (Peprotech) for one day and cocultured with 1×10⁶ HLA-A2+ PBLs in complete RPMI medium. Unmodified H1-derived DCs (H1-DC) and H1-DCs pulsed by 10 μg/ml MART-1 peptide (ELAGIGILTV) (ProImmune, Oxford, U.K.) for 4 hours were also used as negative and positive controls, respectively. Nine days after coculture, the samples were stained with APC mouse anti-human CD3 (BD Biosciences), FITC-labeled anti-CD8 (ProImmune) and R-PE-labeled A*0201/ELAGIGILTV Pentamer (ProImmune). The MART-1-specific CD8+ T cells were detected using a FACSAria flow cytometer (BD Biosciences).

To expand the MART-1-specific CD8+ T cells, bulk cultures were started with 2×10⁶ H1.ME-DCs and 20×10⁶ PBLs. After incubation for 9 days, the cells were restimulated twice on a weekly basis with H1.ME-DCs at DC:PBL ratio of 1:10. Bulk cultures stimulated with H1-DCs were used as controls. The MART-1-specific CD8+ T cells in the bulk cultures were stained and monitored by the FACSAria flow cytometer.

Flow Cytometry and Allostimulation Assay

To study the phenotype of H1.ME-DCs, the cells were stained with antibodies against CD11c, CD40, CD83, CD86, HLA-DR and HLA-A2 (BD Biosciences) and analyzed with a FACSCalibur flow cytometer (BD Biosciences). To check the phenotype of the MART-1-specific CD8+ T cells after multiple stimulations, the cells were stained with R-PE-labeled A*0201/ELAGIGILTV Pentamer and antibodies against CD8, CD45RA and CD62L (BD Biosciences) before analysis using the FACSAria flow cytometer.

To measure the allostimulatory function of DCs, frozen human peripheral blood pan-T cells were thawed and labelled with Carboxyfluorescein diacetate succinimidyl ester (CFSE; Life Technologies) as described previously [9]. To set up the allostimulation assay, 2×10⁵ CFSE-labelled pan-T cells were co-cultured with DCs at various DC:T cell ratios. After a 5-day incubation, the samples were stained with APC mouse anti-human CD4 antibody (BD Biosciences) and the CD4+ T cell proliferation was evaluated by CFSE dilution after gating on CD4+ population using FACSAria flow cytometer.

ELISPOT and Cytotoxicity Assay

To measure GrB secretion, a Human Granzyme B ELISpot Kit (R&D Systems, Minneapolis, Minn.) has been used. In brief, 10⁵ expanded T cells and 10⁵ MART-1 peptide-pulsed T2 cells were cocultured on a human GrB microplate for 4 hours. GrB spots were then stained as described in the manufacturer's manual and counted using an ImmunoSpot Analyzer (CTL, Shaker Heights, Ohio).

To measure cytotoxicity of the expanded MART-1-specific T cells, a flow cytometry-based VITAL-FR assay was employed [13]. In brief, T2 cells stained with CFSE and pulsed with MART-1 peptide were used as specific target cells, while CFSE-stained T2 cells pulsed with HLA-A2-restricted WT1 peptide (WT1126-134, RMFPNAPYL [SEQ ID NO: 3]; ProImmune) were used as non-specific target cells. T2 cells stained with Far Red DDAO-SE (FR; Life Technologies) and pulsed with gp120 peptide (HIV-1 env gp12090-98, KLTPLCVTL [SEQ ID NO: 4]; ProImmune) were used as internal control target cells. After multiple stimulations with H1.ME-DCs or H1-DCs, PBLs were cocultured with 4×10⁴ target cells and 4×10⁴ internal control target cells at the indicated effector: target (E:T) ratios. Cocultures of target cells and internal control target cells without effector cells were used for comparison. After overnight incubation, all samples were assessed by FACSAria flow cytometer and the % of specific lysis at each E:T ratio was calculated as following: % of specific lysis=[1−(# of target cells/# of internal control target cells)_{for an E:T ratio}/(# of target cells/# of internal control target cells)_{without effectors}]×100%.

To measure cytotoxicity of the expanded MART-1-specific T cells, a flow cytometry-based VITAL-FR assay was employed [13]. In brief, T2 cells stained with Carboxyfluorescein diacetate succinimidyl ester (CFSE; Life Technologies) and pulsed with MART-1 peptide were used as specific target cells, while T 2 cells stained with Far Red DDAO-SE (FR; Life Technologies) and pulsed with a gp120 peptide (HIV-1 env _{gp12090-98}, KLTPLCVTL; ProImmune) were used as control target cells. The expanded MART-1-specific T cells were cocultured with 4×10⁴ specific target cells and 4×10⁴ control target cells at the indicated effector: target (E:T) ratios. Cocultures of specific target cells and control target cells without effector cells were used as controls. After overnight incubation, all samples were assessed by the FACSAria flow cytometer and the % of specific lysis at each E:T ratio was calculated as following: % of specific lysis=[1−(# of specific target cells/ # of control target cells)for an E:T ratio/(# of specific target cells/# of control target cells)without effectors]×100%.

Statistics

The statistical significance of differences was determined by two-sided Student's t-test. A p value of <0.05 was considered to be statistically significant.

Results

Tumor Antigen Gene-Modified hPSCs Produce Tumor Antigen-Expressing DCs

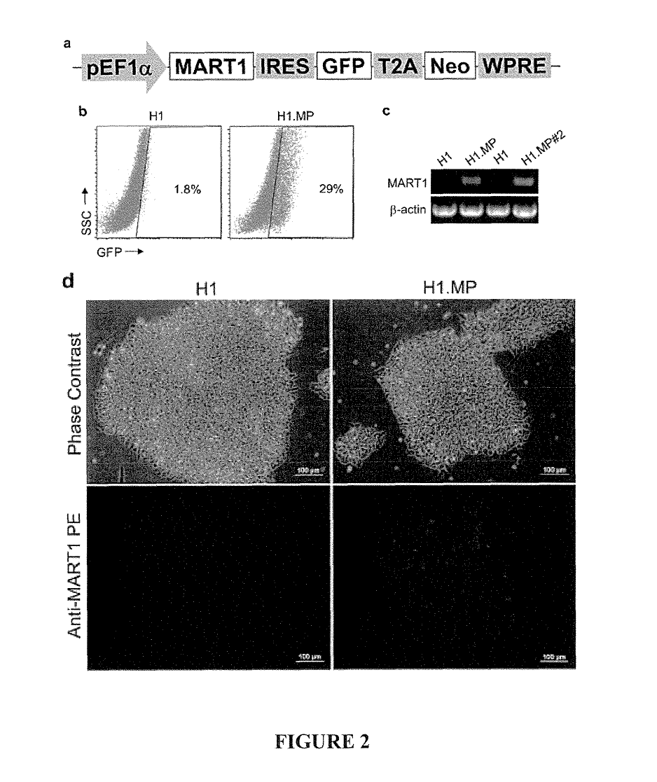

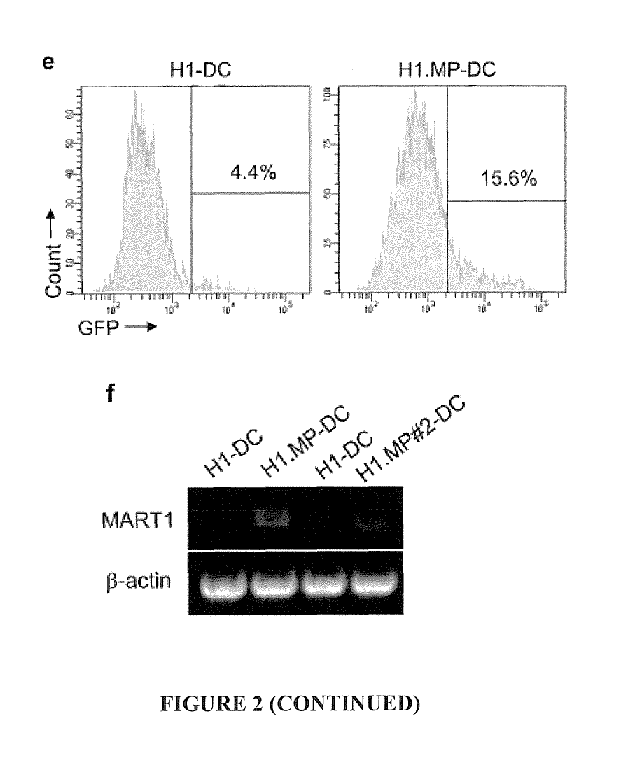

To investigate whether hPSCs can be modified by a tumor antigen gene and subsequently used to derive tumor antigen-expressing DCs, we generated a lentivector carrying a MART-1 gene, designated as LV.MP (FIG. 2a). LV.MP also contains a GFP gene as reporter and a neomycin-resistance gene for drug selection (FIG. 2a). This lentivector was used to transduce an hPSC line, H1. After selection with G418, G418-resistant H1 lines were generated. One of these lines, H1.MP showed substantial GFP expression (FIG. 2b). Moreover, MART-1 expression was also observed in H1.MP as demonstrated at both RNA level (FIG. 2c) and protein level (FIG. 2d). Both H1.MP line and parental H1 line were then used to generate DCs, designated as H1.MP-DCs and H1-DCs, respectively. Although both GFP and MART-1 were still expressed in H1.MP-DCs, the expression levels were low (FIG. 2e, FIG. 2f). These results indicate that it is feasible to derive tumor antigen-expressing DCs from tumor antigen gene-modified hPSCs. However, a further increase of tumor antigen expression level in these DCs may increase antigen presentation on the DC surface.

DCs Derived from Tumor Antigen Gene-Modified hPSCs Present Tumor Antigen

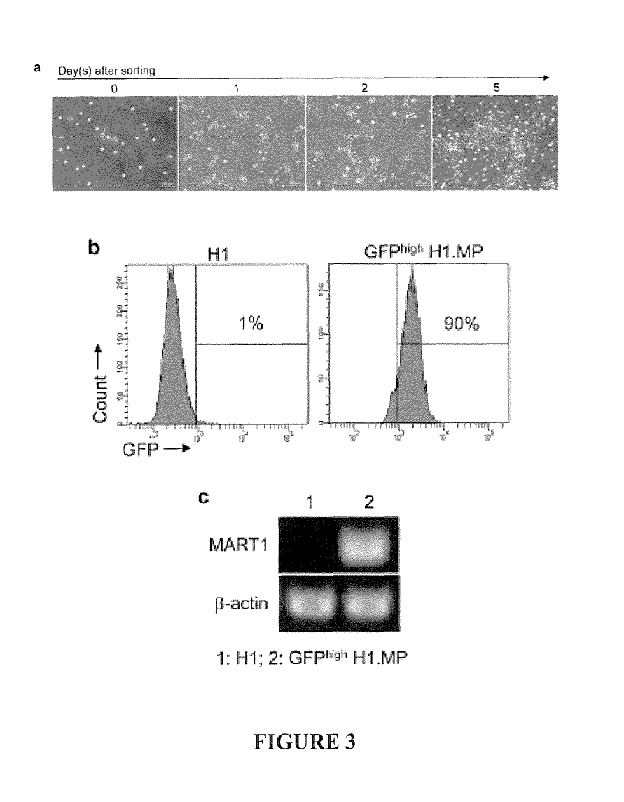

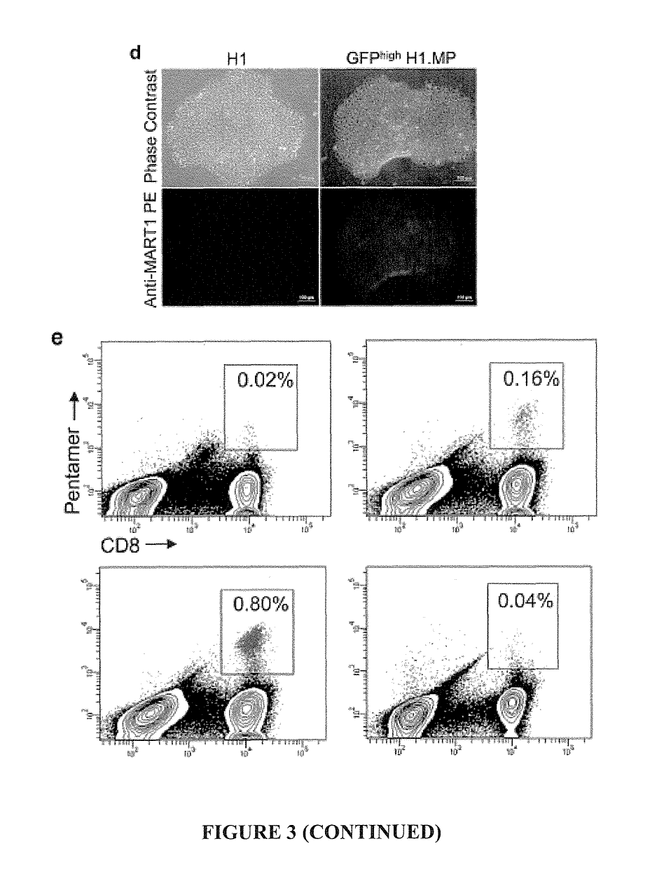

To obtain higher levels of tumor antigen expression in hPSC-DCs, the GFP^{high} H1.MP cells were enriched by fluorescence-activated cell sorting. These GFP^{high} H1.MP cells survived the cell sorting process as demonstrated by cell proliferation after sorting (FIG. 3a). The resulting H1 cell line not only showed a high percentage of GFP+ cells (FIG. 3b), but also an enhanced MART-1 expression as demonstrated by both RT-PCR (FIG. 3c) and immunostaining (FIG. 3d). Using these GFP^{high} H1.MP cells, we derived DCs and checked their tumor antigen presentation. As shown in FIG. 3e, these GFP^{high} H1.MP-derived DCs efficiently expanded the primed MART-1-specific CD8+ T cells during a restimulation process. However, we also observed that there was no specific T cell response while using these DCs for priming. This suggests that tumor antigen is expressed, processed and presented by these hPSC-DCs. For some antigens, the antigen presentation level may need to be increased in order to prime a T cell response.

Modification of hPSCs with Tumor Antigen Epitope-Coding Minigene

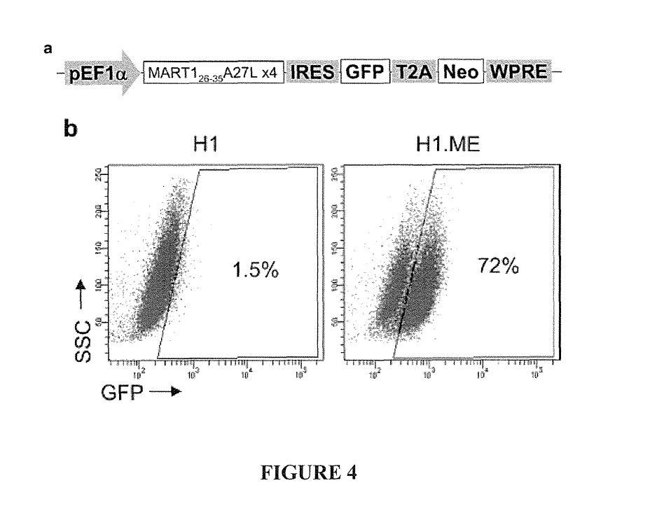

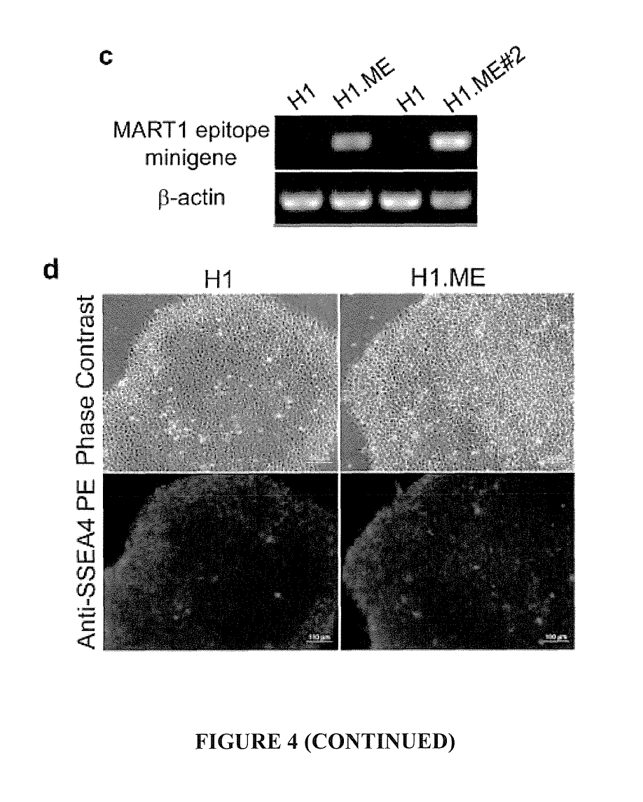

In addition to tumor antigen expression level, the tumor antigen processing efficiency by DCs is equally crucial for tumor antigen presentation on DC surface. It is well studied that some tumor antigens including MART-1 are poorly processed by immunoproteasomes of DCs [14]. To facilitate the MART-1 antigen processing and thus to enhance MART-1 antigen presentation on hPSC-DCs, we generated another lentivector, LV.ME to antigenically modified H1 cells (FIG. 4a). Instead of carrying the whole MART-1 gene, this LV.ME carries a minigene that contains an ubiquitin sequence for proteasomal targeting and a sequence of four MART-1 epitopes to facilitate antigen processing as well as to increase antigenic epitope copy number. This lentivector was able to efficiently modify H1 cells as demonstrated by significant GFP expression in a resulting cell line, H1.ME (FIG. 4b). RT-PCR results indicated that the minigene was also expressed in these H1.ME cells (FIG. 4c). Furthermore, such genetic modification using minigene did not affect “stem cell” status as indicated by the typical hPSC morphology and SSEA-4 expression in H1.ME cells (FIG. 4d).

Tumor Antigen Epitope-Coding Minigene is Expressed in DCs Derived from Minigene-Modified hPSCs

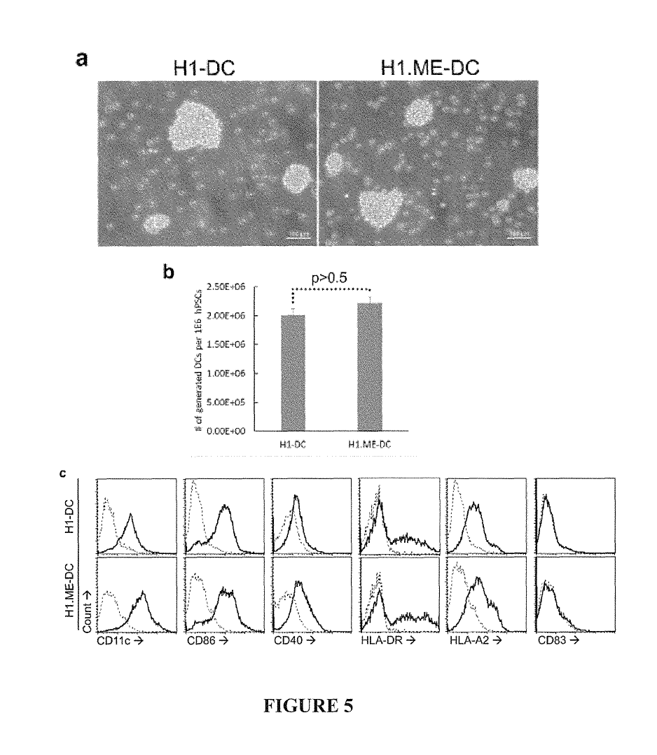

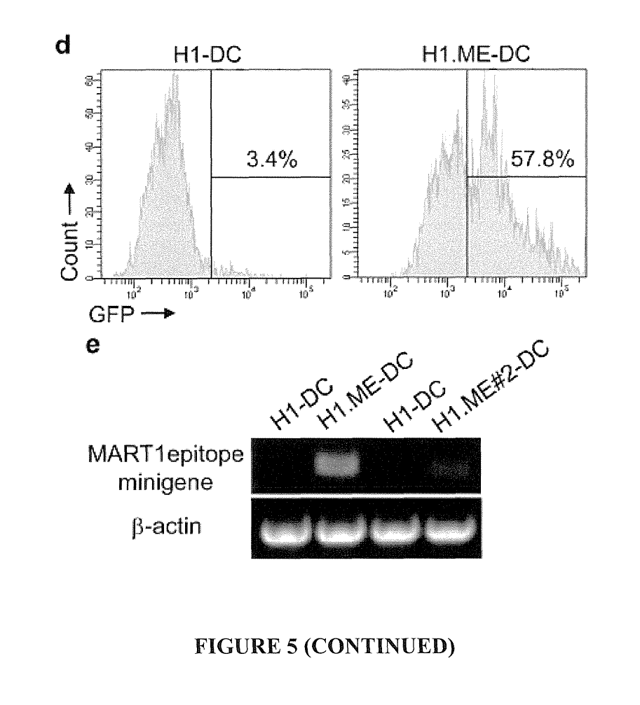

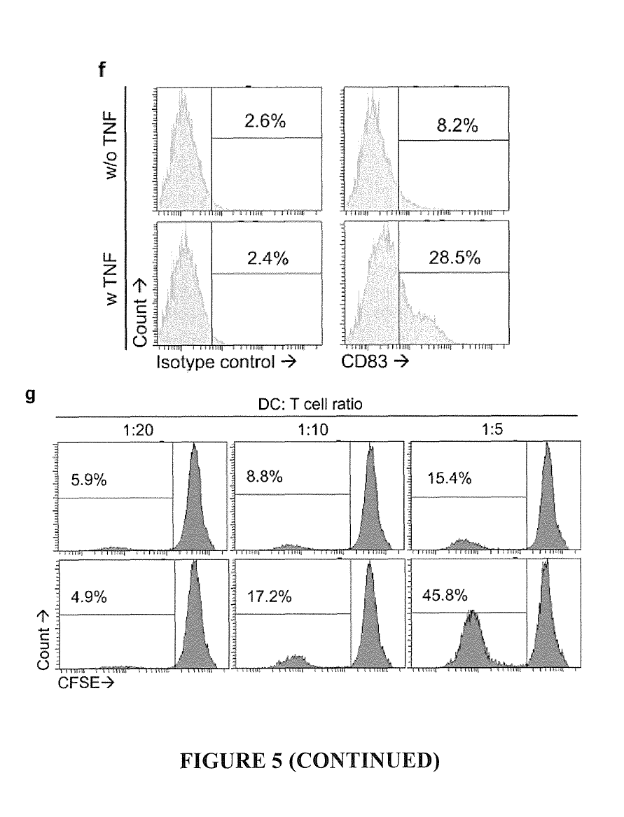

To investigate whether the minigene-modified hPSCs can still generate DCs, and moreover, whether such generated DCs still express the tumor antigen epitope-coding minigene, we derived DCs from H1.ME cells using a three-step protocol as previously described [9, 10, 12]. The resulting H1.ME-DCs were similar in morphology (FIG. 5a) and phenotype (FIG. 5c) to DCs derived from unmodified H1 cells. The modified H1.ME-DCs expressed typical DC surface markers like CD11c, CD86, CD40 and HLA-DR, but little CD83 (FIG. 5c), which suggests an immature DC phenotype. The modified H1.ME-DCs also expressed HLA-A2 (FIG. 5c), a MHC class I molecule that is important for MART-1 epitope presentation in this study. The yield of DCs from H1.ME cells also resembled that from H1 cells (FIG. 5b). In terms of transgene expression, more than half of these H1.ME-DCs remained GFP+ as measured by flow cytometry (FIG. 5d); more importantly, obvious minigene expression was detected by RT-PCR in these H1.ME-DCs (FIG. 5e). These results suggest that minigene-modified hPSCs are able to differentiate into minigene-expressing DCs. For further maturation, H1.ME-DCs were cultured with 20 ng/ml TNF for one day. This TNF-treatment up-regulated the CD83 expression on H1.ME-DCs (FIG. 5f) and improved their allostimulatory function on CD4+ T cells (FIG. 5g), suggesting the immunogenic property of these DCs.

DCs Derived from Minigene-Modified hPSCs Efficiently Prime Tumor Antigen-Specific T Cell Response

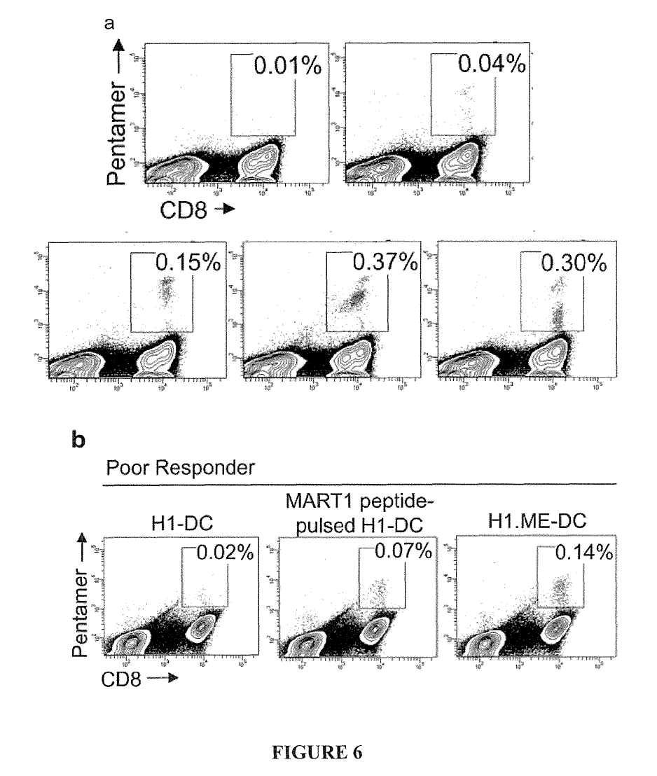

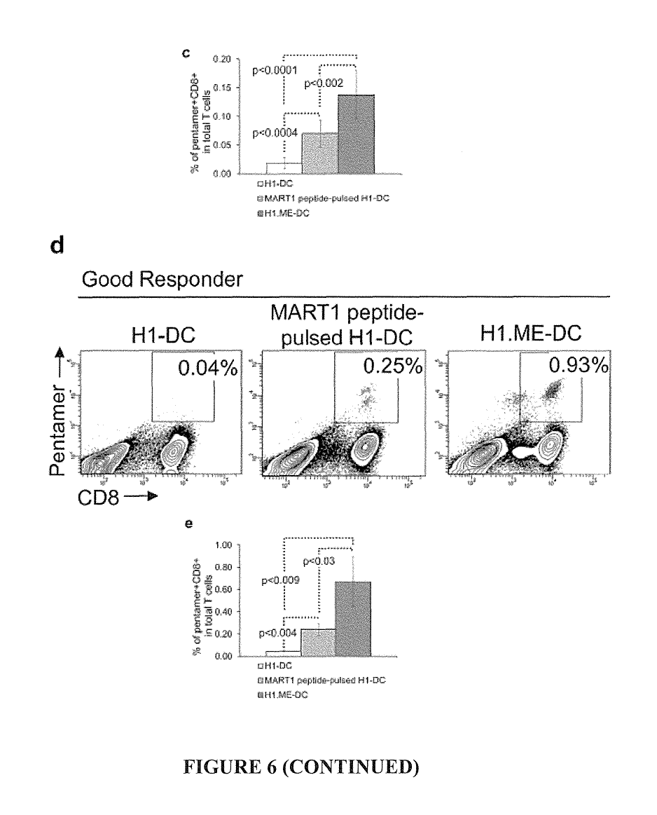

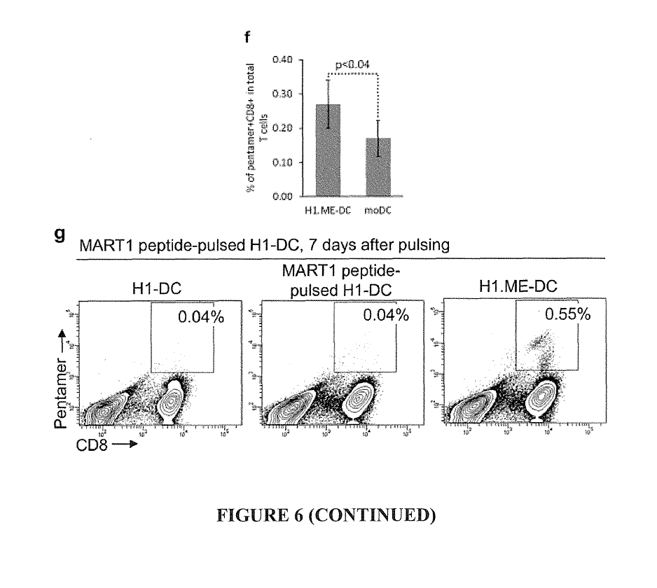

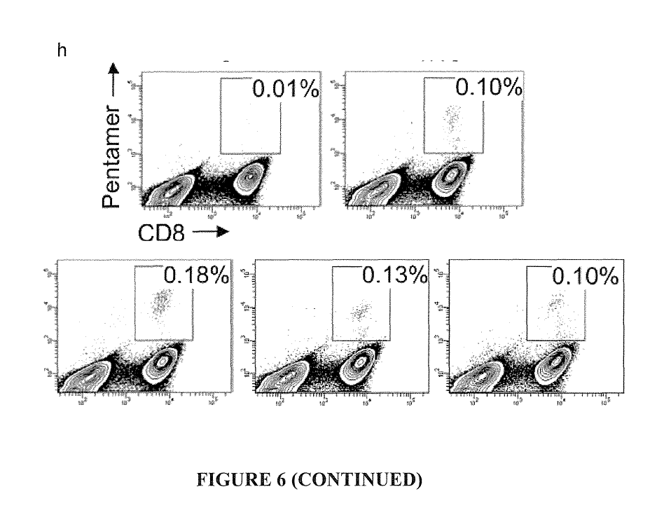

To examine whether the expression products of tumor antigen epitope-coding minigene can be efficiently processed and presented in DCs derived from the minigene-modified hPSCs, we assessed the ability of H1.ME-DCs to prime a MART-1-specific CD8+ T cell response and compared its efficacy to that of H1-DCs pulsed with 10 μg/ml MART-1 peptide, which is an optimal peptide concentration to load h1-DCs (FIG. 6a). H1.ME-DCs were cocultured with HLA-A2+ peripheral blood lymphocytes (PBLs) from healthy donors. Nine days later, MART-1-specific T cells were identified by pentamer staining. As shown with PBLs of low responsiveness, H1.ME-DCs efficiently primed a MART-1-specific T cell response and the efficacy was significantly better than that of the MART-1 peptide-pulsed H1.DCs, which were prepared with a commonly used antigen-loading approach (FIG. 6a and FIG. 6b). Similar results were obtained using PBLs of high responsiveness (FIG. 6d and FIG. 6e), which further confirmed that the minigene products were efficiently processed in H1.ME-DCs and the resulting tumor antigen epitopes were sufficiently presented on H1.ME-DCs for T cell priming. Moreover, such produced H1.ME-DCs were more efficient than the commonly used moDCs pulse with MART-1 peptide (FIG. 6f).

The sustainability of MART-1 epitope presentation in these two types of DCs were compared side by side. After a 4-hour peptide pulsing, the MART-1 peptide-pulsed H1-DCs were washed and further cultured for 7 days before use for priming. Unpulsed H1-DCs and H1.ME-DCs were employed as controls. After this prolonged culture, the MART-1 peptide-pulsed H1-DCs no longer induced a specific T cell response; in contrast, H1.ME-DCs maintained T cell priming ability (FIG. 6g). This result indicates that H1.ME-DCs have more sustainable MART-1 antigen presentation than the MART-1 peptide-pulsed H1-DCs, wherein the former may be continuously supplied with MART-1 epitope from minigene expression. To explore the possible benefit of using high dosage of DCs in T cell priming, cocultures were set up using H1.ME-DCs and HLA-A2+ PBL at various DC:PBL ratios (FIG. 6h). The results showed that H1.ME-DCs were able to induce a specific T cell response with a wide range of DC:PBL ratio, although no increased benefit was seen at ratios greater than 1:5.

CTLs Expanded by DCs Derived from Minigene-Modified hPSCs are Immunocompetent

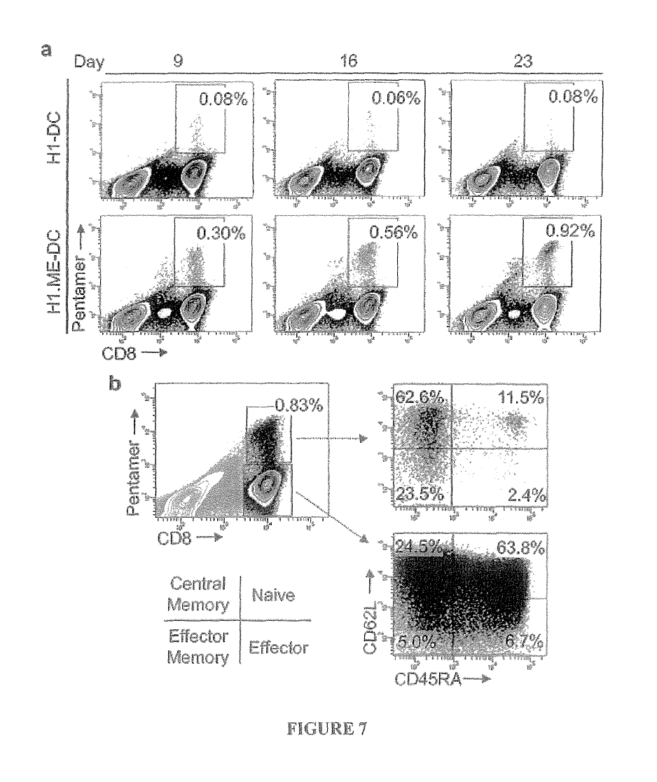

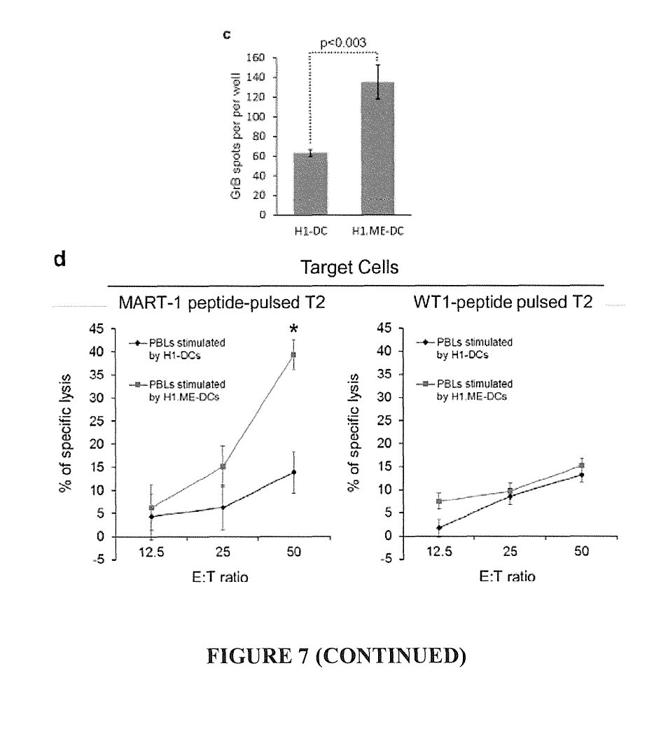

To test whether H1.ME-DCs were able to expand specific cytotoxic T lymphocytes (CTLs) in bulk culture, HLA-A2+ PBLs were first primed and then restimulated twice with H1.ME-DCs. The MART-1-specific T cell expansion during this process was monitored by pentamer staining. As shown in FIG. 7a, the MART-1-specific T cell population continued to increase after each stimulation with H1.ME-DCs, but not with H1-DCs. Interestingly, these expanded MART-1-specific CTLs predominantly possessed central memory and effector memory phenotypes (FIG. 7b), which correlate with the less differentiated T cell populations that have better antitumor immunity. To test the function of these expanded CTLs, the secretion of granzyme B (GrB) was detected by ELISPOT. As shown in FIG. 7c, the CTLs expanded by H1.ME-DCs were responsive to stimulation by MART-1 peptide-pulsed T2 cells. Furthermore, these CTLs were not functionally exhausted after multiple stimulations. They were able to specifically kill target cells as demonstrated by the cytotoxicity assay (FIG. 7d). These results suggest that H1.ME-DCs are competent antigen-presenting cells for specific CTL expansion.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

As used in this specification and the appended claims, the singular forms “a”, “an” and “the” include plural reference unless the context clearly dictates otherwise. As used in this specification and the appended claims, the terms “comprise”, “comprising”, “comprises” and other forms of these terms are intended in the non-limiting inclusive sense, that is, to include particular recited elements or components without excluding any other element or component. As used in this specification and the appended claims, all ranges or lists as given are intended to convey any intermediate value or range or any sublist contained therein. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the scope of the invention.

### References

1. Galluzzi L, Senovilla L, Vacchelli E, et al. Trial watch: Dendritic cell-based interventions for cancer therapy. Oncoimmunology. 2012; 1: 1111-1134.

2. Vacchelli E, Vitale I, Eggermont A, et al. Trial watch: Dendritic cell-based interventions for cancer therapy. Oncoimmunology. 2013; 2: e25771.

3. Butterfield L H. Dendritic cells in cancer immunotherapy clinical trials: are we making progress? Front Immunol. 2013; 4: 454.

4. Gilboa E. DC-based cancer vaccines. J Clin Invest. 2007; 117: 1195-1203.

5. Nicolette C A, Healey D, Tcherepanova I, et al. Dendritic cells for active immunotherapy: optimizing design and manufacture in order to develop commercially and clinically viable products. Vaccine. 2007; 25 Suppl 2: B47-60.

6. Wimmers F, Schreibelt G, Skold A E, et al. Paradigm Shift in Dendritic Cell-Based Immunotherapy: From in vitro Generated Monocyte-Derived DCs to Naturally Circulating DC Subsets. Front Immunol. 2014; 5: 165.

7. DeFrancesco L. Landmark approval for Dendreon's cancer vaccine. Nat Biotechnol. 2010; 28: 531-532.

8. Senju S, Matsunaga Y, Fukushima S, et al. Immunotherapy with pluripotent stem cell-derived dendritic cells. Semin Immunopathol. 2011; 33: 603-612.

9. Zeng J, Shahbazi M, Wu C, et al. Enhancing immunostimulatory function of human embryonic stem cell-derived dendritic cells by CD1d overexpression. J Immunol. 2012; 188: 4297-4304.

10. Zeng J, Wang S. Human dendritic cells derived from embryonic stem cells stably modified with CD1d efficiently stimulate antitumor invariant natural killer T cell response. Stem Cells Transl Med. 2014; 3: 69-80.

11. Nierkens S, Janssen E M. Harnessing dendritic cells for tumor antigen presentation. Cancers (Basel). 2011; 3: 2195-2213.

12. Slukvin, I I, Vodyanik M A, Thomson J A, et al. Directed differentiation of human embryonic stem cells into functional dendritic cells through the myeloid pathway. J Immunol. 2006; 176: 2924-2932.

13. Stanke J, Hoffmann C, Erben U, et al. A flow cytometry-based assay to assess minute frequencies of CD8+ T cells by their cytolytic function. J Immunol Methods. 2010; 360: 56-65.

14. Morel S, Levy F, Burlet-Schiltz O, et al. Processing of some antigens by the standard proteasome but not by the immunoproteasome results in poor presentation by dendritic cells. Immunity. 2000; 12: 107-117.

15. Chen D S, Mellrnan I. Oncology meets immunology: the cancer-immunity cycle.

Immunity. 2013; 39: 1-10.

16. Haruta M, Tomita Y, Yuno A, et al. TAP-deficient human iPS cell-derived myeloid cell lines as unlimited cell source for dendritic cell-like antigen-presenting cells. Gene Ther. 2013; 20: 504-513.

17. Senju S, Suemori H, Zembutsu H, et al. Genetically manipulated human embryonic stem cell-derived dendritic cells with immune regulatory function. Stem Cells. 2007; 25: 27202729.

18. Su Z, Frye C, Bae K M, et al. Differentiation of human embryonic stem cells into immunostimulatory dendritic cells under feeder-free culture conditions. Clin Cancer Res. 2008; 14: 6207-6217.

19. Tseng S Y, Nishimoto K P, Silk K M, et al. Generation of immunogenic dendritic cells from human embryonic stem cells without serum and feeder cells. Regen Med. 2009; 4: 513-526.

20. Robson N C, Hoves S, Maraskovsky E, et al. Presentation of tumor antigens by dendritic cells and challenges faced. Curr Opin Immunol. 2010; 22: 137-144.

21. Wilson N S, Villadangos J A. Regulation of antigen presentation and cross-presentation in the dendritic cell network: facts, hypothesis, and immunological implications. Adv Immunol. 2005; 86: 241-305.

22. Smits E L, Anguille S, Cools N; et al. Dendritic cell-based cancer gene therapy.

Hum Gene Ther. 2009; 20: 1106-1118.

23. Breckpot K, Heirman C, Neyns B, et al. Exploiting dendritic cells for cancer immunotherapy: genetic modification of dendritic cells. J Gene Med. 2004; 6: 1175-1188.

24. Kreiter S, Diken M, Selmi A, et al. Tumor vaccination using messenger RNA:

prospects of a future therapy. Curr Opin Immunol. 2011; 23: 399-406.

25. Fontes A, Lakshmipathy U. Advances in genetic modification of pluripotent stem cells. Biotechriol Adv. 2013; 31: 994-1001.

26. Kennedy, R. & Celis, E. Multiple roles for CD4+ T cells in anti-tumor immune responses. Immunological reviews 2008; 222: 129-144.

27. Liechtenstein, T., Perez-Janices, N. & Escors, D. Lentiviral vectors for cancer immunotherapy and clinical applications. Cancers (Basel). 2013; 5: 815-837.

28. Gay, T., Gersbach, C. A. & Barbas, C. F., 3rd ZFN, TALEN, and CRISPR/Cas-based methods for genome engineering. Trends In Biotechnology. 2013; 31: 397-405.

29. Sadelain, M., Papapetrou, E. P. & Bushman, F. D. Safe harbours for the integration of new DNA in the human genome. Nature Reviews. Cancer. 2012; 12: 51-58.

30. Takahashi and Yamanaka (2006) Cell 126:663-676.

31. Silk, K. M., Tseng, S. Y., Nishimoto, K. P., Lebkowski, J., Reddy, A., Fairchild, P. J. Differentiation of dendritic cells from human embryonic stem cells. Methods in Molecular Biology. 2011; 767: 449-461.

1-31. (canceled)

32. A method of loading antigen in a dendritic cell for antigen presentation, the method comprising:
modifying a pluripotent stem cell with a nucleic add molecule encoding a proteosomal targeting sequence and one or more immunogenic epitopes of an antigen under control of a constitutively active promoter;
inducing the pluripotent stem cell to differentiate into a dendritic cell that expresses and presents the one or more immunogenic epitopes for antigen presentation by an MHC 1 molecule in the dendritic cell.

33. The method of claim 32, wherein the pluripotent stem cell is an induced pluripotent stem cell.

34. The method of claim 32, wherein modifying comprises transducing using a viral or nonviral method to deliver the nucleic acid molecule into the pluripotent stem cell.

35. The method of claim 34, wherein the modifying comprises transducing the pluripotent stem cell with a retroviral vector.

36. The method of claim 35, wherein the retroviral vector is a lentiviral vector.

37. The method of claim 32, wherein the pluripotent stem cell is a mammalian cell,

38. The method of claim 37, wherein the pluripotent stem cell is a human cell.

39. The method of claim 32, wherein the one or more immunogenic epitopes of the antigen is a tumor immunogenic epitope, a viral immunogenic epitope, a bacterial Immunogenic epitope or an autoimmune disease immunogenic epitope.

40. The method of claim 32, wherein each of the more immunogenic epitopes encoded by the nucleic add molecule have a same amino add sequence.

41. The method of claim 32, wherein each of the more immunogenic epitopes encoded by the nucleic add molecule have a different amino add sequence.

42. The method of claim 32, wherein the proteosomal targeting sequence is a ubiquitin sequence.

43. The method of claim 40, wherein each of the more immunogenic epitopes are separated by a spacer sequence.

44. A dendritic cell that is derived from a pluripotent stem cell, the pluripotent stem cell stably modified with a nucleic acid molecule encoding a proteosomal targeting sequence and one or more immunogenic epitopes of an antigen under control of a constitutively active promoter, wherein the dendritic cell expresses and presents the one or more immunogenic epitopes for antigen presentation by an MHC 1 molecule in the dendritic cell,

45. The dendritic cell of claim 44, wherein the cell expresses one or more of CD11c, CD86 and HLA.

46. A vaccine comprising a dendritic cell that is derived from a pluripotent stem cell, the pluripotent stem cell stably modified with a nucleic acid molecule encoding a proteosomal targeting sequence and one or more immunogenic epitopes of an antigen under control of a constitutively active promoter, wherein the dendritic cell expresses and presents the one or more immunogenic epitopes for antigen presentation by an MHC 1 molecule in the dendritic cell.

47. A method of inducing an immune response in a subject, the method comprising:
administering a dendritic cell that is derived from a pluripotent stem cell, the pluripotent stem cell stably modified with a nucleic acid molecule encoding a proteosomal targeting sequence and one or more immunogenic epitopes of an antigen under control of a constitutively active promoter, wherein the dendritic cell expresses and presents the one or more immunogenic epitopes for antigen presentation by an MHC 1 molecule in the dendritic cell, to a subject in need of immunity to the antigen.

48. The method of claim 47, wherein the immune response is a T-cell mediated immune response.

49. The method of claim 47, wherein the dendritic cell is autologous with the subject.

50. The method of claim 47, wherein the dendritic cell is allogeneic with the subject.

51. The method of claim 47, wherein the subject is in need of treatment for cancer and the antigen is a tumour antigen.
